(19) United States
(12) Patent Application Publication
(10) Pub. No.: US 2012/0164217 A1
(43) Pub. Date: Jun. 28, 2012

(54) SCENTED CAPSULES

(76) Inventors: Dominique Nicolas Cade, Colmar (FR); Claire Genevieve Odile Tardy, Colmar (FR)

(21) Appl. No.: 13/394,034

(22) PCT Filed: Aug. 24, 2010
(86) PCT No.: PCT/IB10/53798

§ 371 Date: Mar. 2, 2012

Related U.S. Application Data
(60) Provisional application No. 61/239,819, filed on Sep. 4, 2009.

(51) Int. Cl. A61K 9/48

(52) U.S. Cl. 424/452

(57) ABSTRACT

The present invention relates to new scented hard capsules, a process for their manufacture and use of such capsules particularly but not exclusively for oral administration to humans or animals of products such as pharmaceuticals or cosmetics.

### FIELD OF THE INVENTION

The present invention relates to new scented capsules, a process for their manufacture and use of such capsules particularly but not exclusively for oral administration to humans or animals of products such as pharmaceuticals or cosmetics.

### BACKGROUND OF THE INVENTION

Soft and hard capsules are widely used as oral administration forms for humans and animals e.g. in cosmetic or pharmaceutical fields. In this context, it is often desirable to impart particular scents and/or non persisting flavours to the capsules. For example, masking the unpleasant mouth feeling of some film-forming polymers used in capsule manufacturing improves the compliance of final users. Similarly, it is predicted that capsules possessing animal-food like scents could be more easily administered to animals.

Conventionally, the problem of imparting scents or flavours to a capsule has been tackled by directly admixing aromatic substances to the shell formula of the capsules before their manufacture so that the aromatic substances are part of the finished capsule shell. Examples of this procedure can be found in U.S. 20060078608, U.S. 20060222699 and WO 2007054853. However, this technical solution presents some relevant drawbacks. For example, the risk of chemical interactions (e.g. cross-linking) between shell film-forming polymer(s) or encapsulated substances and the flavours is significantly increased. As a consequence, key physical properties of the capsules such as dissolution and disintegration profiles may be unpredictably and drastically modified. Additionally, devices used in capsule manufacturing could remain contaminated with the traces of flavouring substances used then requiring time-consuming and expensive cleaning procedures to avoid contamination of subsequently manufactured non-scented batches. Finally, large quantities are needed to compensate evaporation during capsules manufacture.

Another known technical solution is providing capsules with an external—often sugar-based—thick and rigid, enrobing flavoured shell so as to obtain a confectionary-like product. For example, WO03045166 discloses a capsule comprising a core and a coating made of film-forming polymer(s). In example 4 of WO031045166 a confectionery product for oral hygiene is produced by enrobing seamless gelatine in a coating turbine using maltitol, gum arabic, shellac gum, vegetable oil, titanium dioxide and a menthol-flavouring powder. A typical drawback of this technique is that it requires the capsules to be manufactured and filled before being scented since the coating does not allow re-opening of pre-locked but empty capsules.

Alternatively, it is also known to apply flavouring substances on capsule surfaces by e.g. dipping or surface spraying the capsules with flavour-containing solutions and subsequently drying the capsules, typically by air. U.S. Pat. No. 3,529,043 discloses soft gelatin capsules for medical usage that are dyed on the surface by adding a volatile dye solution to the already formed soft capsules. The dye is absorbed on the surface of the capsules to give the colour. A water soluble flavour or perfume can be added to the dye solution to impart a flavour or odor to the capsules. Air is blown over the tumbling capsules to remove volatile solvents therefrom. Similarly, WO2001067887 discloses a gelatine or alginate capsule filled with a water-based mixture of a pigment and fish oil. After the production, the capsule is either dipped into a bath wherein suitable taste substances are added, or surface sprayed with a solution of taste substances, and subsequently dried in the air.

Drawbacks commonly linked for this technique are for example (i) the risk of modifying the chemical composition of capsule shells, especially when using liquids that are able to partially solubilise the polymer of the capsule shell; (ii) significant loss of flavours that typically occurs during the process entails increased production costs; (iii) the final drying step can easily affect the moisture content of capsules shell thus entailing a modification of capsule brittleness. All these drawbacks, although being tolerated in less-demanding fields such as confectionary, are totally unacceptable in case of products for uses that are characterized by strict regulatory boundaries such as administration of cosmetics and pharmaceuticals.

None of the technical solutions indicated above thus fully meets the need for scented capsules, particularly for conventional oral administration to humans or animals of products such as cosmetics or pharmaceuticals, where the capsules can be easily manufactured without excessive costs and wherein capsule physical-chemical properties are not imparted during production so as to guarantee safety of use and dissolution/disintegration properties in accordance with health regulations.

Another desirable goal would be to provide the possibility to use a wide range of scenting agents without incurring the risk of chemical interactions between the scenting agents and the film-forming polymer(s) of the capsule shell as well as the encapsulated substances.

Another desirable goal would be to provide a cost-effecting process for the manufacture of scented capsules that does not require expensive and complicated post-manufacturing cleaning procedures to remove potential contamination of scenting agents and that allows minimizing flavour(s) losses during production.

### SUMMARY OF THE INVENTION

The above and other objects are achieved by a scented hard capsule which is externally coated with a scenting composition, wherein said composition comprises one of more scenting agents and one or more gliding enhancing agents and wherein said composition is in powder form or oil form at about room temperature.

The above and other objects are achieved by a method for the manufacture of a scented hard capsule as defined above, wherein said method comprises a step of contacting one or more capsules with a scenting composition as defined above.

The above and other objects are achieved by a scented hard capsule obtainable by a method as defined above, wherein said capsule is suitable for use in oral administration of one or more encapsulated products to a human or an animal being.

The above and other objects are achieved by the use of a scented hard capsule as defined above for the manufacture of a dosage form for oral administration of one or more encapsulated products to a human or animal being.

### DETAILED DESCRIPTION OF THE INVENTION

In the present invention, a scent is imparted to hard capsules substantially by coating the external surface of capsule with a scenting composition as defined below. Such composition is applied onto, and preferably in direct contact with the external surface of the capsules. This means that no intermediate layers are preferably interposed between the capsules external surface and the scenting composition. Unless otherwise indicated, the external surface is the surface of the capsules that is not in contact with the encapsulated substance(s).

The scent may be perceived by a final user through his/her olfactory system as a perfume for example when opening a bottle containing the capsules in bulk or after removing the capsules from the blister. However, as the olfactory system is involved in the perception of flavours as well, it must be understood that the presence of scenting agents may also be perceived by a final user as a non persisting flavour once a capsule is put in his/her mouth.

Unless otherwise indicated, the wording “capsule” or “capsules” refers to hard capsules (i.e. capsules having a hard polymer shell; also known as hard shell capsules). In a preferred embodiment, the capsules of the invention are pharmaceutical hard capsules, more preferably empty (i.e. capsule shells) or already filled hard pharmaceutical capsules, and even more preferably empty hard pharmaceutical capsules.

The hard capsules of the present invention do not structurally depart from the conventional definition of hard capsules. They normally comprise two co-axial, telescopically-joined parts, referred to as body and cap defining a shell. Normally, caps and bodies have a side wall, an open end and a closed end. The length of the side wall of each of said parts is generally greater than the capsule diameter. The shell of the hard capsules of the invention can be manufactured with conventional techniques as for example by using a dip-molding process. In case the substances to be encapsulated are in liquid form, it is intended that the hard capsules of the invention may be sealed or banded according to conventional techniques.

The shell of the capsules of the invention mainly comprises one or more film-forming polymers that are well-known in the art. Film-forming polymers are preferably selected from the group consisting of gelatin, polyvinyl alcohol, starch, starch derivatives (for example hydroxypropylated starch), cellulose, cellulose derivatives, preferably HPMC, pullulan and mixtures thereof. In one embodiment, the film forming polymers comprise one or more of HPMC, gelatin, starch derivatives and pullulan. In one embodiment, the film forming polymers comprise one or more of gelatin and HPMC. In one embodiment, the film forming polymers comprise gelatin. In one embodiment, the film forming polymers comprise HPMC. In one embodiment, the HPMC of the invention, the HPMC of the invention comprises a HPMC grade 2910 as defined in USP30-NF25. In one embodiment, the HPMC of the invention, the HPMC of the invention comprises a HPMC grade 2906 as defined in USP30-NF25. In one embodiment, HPMC of the invention is a HPMC having a methoxy content of 27.0-30.0% (w/w), a hydroxypropoxy content of 4.0-7.5% (w/w) and a viscosity of 3.5-6.0 cPs as a 2% weight solution in water at 20° C. (e.g. a HPMC grade 2906 as defined in USP30-NF25). Hard capsules obtained using this specific type of HPMC are for example disclosed in PCT/IB07/003160 and are also commercially available. Methoxy and hydroxypropoxy contents are expressed according to the USP30-NF25. The use of this specific HPMC is allows for example avoiding the use of setting systems (see additional ingredients below) to obtain hard capsules by dip moulding processes.

In one embodiment, the shell of the capsules of the invention comprise gelatin and does not comprise cellulose or cellulose derivatives like HPMC, starch, modified starch, polyvinyl alcohol or its derivatives, and pullulan.

In one embodiment, the shell of the capsules of the invention comprise HPMC and does not comprise cellulose derivatives other than HPMC, gelatin, starch, modified starch, polyvinyl alcohol or its derivatives, and pullulan. In this embodiment, the shell of the capsules of the invention may comprise one or more setting systems as defined below. In one embodiment, the shell of the capsules of the invention comprise HPMC, preferably a HPMC grade 2910 as defined in USP30-NF25, and one or more setting systems. In one embodiment, the shell of the capsules of the invention comprise HPMC, preferably a HPMC grade 2906 as defined in USP30-NF25, but does not contain any setting systems.

Unless otherwise indicated, in the present invention the wording “hard gelatin capsule” or “hard HPMC capsule” refers to a hard capsule wherein gelatin or HPMC are the only one or main capsule shell-constituting film forming polymer by weight. Preferably, a hard gelatin capsule comprises gelatin and does not comprise other film forming polymers as indicated above. Preferably, a hard HPMC capsule comprises HPMC and does not comprise other film forming polymers as indicated above.

Beside the film-forming polymer(s), the capsule shells of the invention may contain additional ingredients that are typically used in capsule manufacturing and that are well-known to a skilled person in the art.

For example, one or more plasticizers, such as glycerine or propylene glycol can be included in the shell formula to optimize the physical properties of the capsule shells. Typically, manufacturing soft capsules requires higher amounts of plasticizers than manufacturing hard capsules. The amount and type of plasticizer(s) to be used as well as the physical properties imparted to the resulting capsules are all aspects that can easily be determined by a skilled person relying on its common general knowledge.

Other common components of capsule shells are for example the so-called setting systems. Setting systems are typically used to optimize the setting ability of many film forming polymers that would set too slowly or would not set at all in conventional dip-moulding process wherein gelation is induced by cooling the film formed on the surface of dipping pins. Typical examples of such polymers are many technical grades of HPMC (e.g. HPMC 2910 as defined in the US Pharmacopoeia) or hydroxypropylated starch. Typically, setting systems contain one or more hydrocolloids (also referred to in the art as gelling agents) and/or one or more cations (also referred to in the art as co-gelling agents or gelling aids or auxiliaries for gelation). Typical hydrocolloids are selected from the group consisting of pectin, agarose, gelatin, gellan, starch xanthan with locust bean gum, xanthan with konjac, alginates, agar gum, guar gum, locust bean gum (carob), carrageenan, welan, rhamsan, furcelleran, curdlan, succinoglycan, scleroglycan, schizophyllan, tamarind gum, dextran, acetan, tara gum, gum arabic, ghatti gum, Khaya grandifolia gum, tragacanth gum, karaya gum, arabian (araban), Konjac mannan, galactomannan, funoran, other exocellular polysaccharides and mixtures thereof. Compatibility of mixtures of hydrocolloids is an aspect well within the common knowledge of a skilled person working in the field of hard shell capsules. The choice of specific hydrocolloid(s) and cations and their respective amounts may depend on the particular film-forming polymer used and the manufacturing technology adopted. These aspects are widely discussed in the art and are well within the common knowledge available to a skilled person in capsule manufacturing. Examples of setting systems are disclosed in EP 1042405 for capsule shells made with fish gelatin; EP 1062274 for capsule shells made with PVA; EP 1117736 for capsule shells made with modified starches; EP 1204699 for capsule shells made with pullulan and U.S. Pat. No. 5,264,223 or U.S. Pat. No. 5,756,123 for capsule shells made with HPMC.

Finally, one or more colouring agents and/or one or more sequestering agents may also be used in the manufacture of capsule shells. In one embodiment, the capsule shells of the invention do not contain any colouring agent and are transparent. In one embodiment, the capsule shells of the invention contain one ore more pharmaceutically or cosmetically acceptable colouring agents.

In the present invention, powder form preferably refers to a mass of loose particles typically having 50% by weight of particles passing through a 100 mesh sieve.

In the present invention, oil form preferably refers to an oily, viscous liquid typically having a viscosity between about 5 and 400 cps, preferably between about 10 and 200 cps, more preferably between about 50 cps and 200 cps at 25° C. as measured with conventional techniques (such as kinematic or dynamic viscosity measuring methods). Powder and oil forms are evaluated at about room temperature. Unless otherwise indicated, in the present invention room temperature is about 25° C.

In a preferred embodiment, the scenting composition comprises one or more scenting agents in an amount between about 0.001 and 1 weight parts, preferably between about 0.02 and 0.5 weight parts, more preferably between about 0.1 and 0.5 weight parts per 1 weight part of the one or more gliding enhancer agents.

In a particularly preferred embodiment, the scenting composition consists of one or more gliding enhancer agents and one or more scenting agents, preferably in the weight ratio indicated above.

Suitable scenting agents in the context of the present invention may be any substance, that typically in liquid form at about room temperature, and that is conventionally used to impart specific flavours or scents to products for human or animal oral consumption. To optimize the scenting effect, it is preferred that said one of more scenting agents be at least partially volatile at about room temperature which preferably means that the scenting agent has a measurable vapour pressure at 25° C. Suitable scenting agents can be selected from the group consisting of anis, apple, apricot, banana, blackcurrant, bubble gum, caramel (Golden syrup), cherry, cherry black, chocolate, cinnamon, coffee, cola, exotic fruits, grapefruit, honey, honey lemon, lemon, lime, mandarin, mango, mint, orange, passion fruit, peach, pear, peppermint, pineapple, raspberry, spearmint, strawberry, tutti frutti, vanilla, beef, chicken, meat, cheese, roast beef juice, and mixtures thereof.

Suitable gliding enhancing agents are substances commercially available and commonly used in capsule manufacturing to improve gliding properties of capsules surface. For example gliding enhancing agents are used to lubricate capsule surfaces, reducing sticking among capsules, facilitating hard capsule filling (in case of pre-assembled empty hard capsules) and facilitating any post-production capsule handling such as packaging. Typically, gliding enhancing agents are applied to capsules e.g. by powder application (e.g. dusting) if in powder form, or spray deposit if in powder or oil form, or droplet deposit if in oil form. Upon application, suitable techniques to distribute the agent over the capsules are e.g. tumbling or shaking or vibrating, or agitating the capsules or a combination of these techniques.

The same application and distribution techniques for conventional gliding enhancing agents are also applicable to distribute the scented composition of the invention.

Gliding enhancer agents are typically commercialized in powder or oil form, wherein the wording “powder or oil form” is as defined above for the scenting composition of the invention. Combinations of one or more of gliding enhancer agents in powder form and one or more of gliding enhancer agents in oil form are also possible.

In a preferred embodiment, the scenting composition is in powder form when at least one of the gliding enhancing agents is in powder form and it represents at least 60% by weight of the total weight of the scenting composition. Gliding enhancer agents in powder form may be selected from the group consisting of SLS (sodium lauryl sulfate), waxes (such as carnauba wax, candelia wax), magnesium stearate, talc, colloidal silica and mixtures thereof. Preferably, the one or more powder gliding enhancer agents comprise, more preferably consist of SLS. Mixtures of SLS and scenting agents are commercially available (for example from Firmenich-Geneva).

Gliding enhancer agents in oil form are typically pharmaceutically or cosmetically acceptable lubricants. In a preferred embodiment, the scenting composition is in oil form when at least one of the one or more gliding enhancing agents is in oil form and it represents at least 60% by weight of the total weight of the composition. Oil gliding enhancer agents may be selected from the group consisting of vegetal oils (such as MCT i.e. medium chain triglycerides), mineral oils (such as paraffin oil), silicone, silicone derivatives (such as dimethylsiloxanes) and mixtures thereof. Preferably, the one or more oil gliding enhancer agents comprise, more preferably consist of, paraffin oil, MCT or mixtures thereof.

Other optional ingredient may be added to the scenting composition. Optional ingredients are for example colorants typically used for products for human or animal oral ingestion.

The scenting composition of the invention can be prepared by numerous techniques known in the art. For example, the scenting composition can be prepared by mixing together the one or more gliding enhancing agents and the one or more scenting agents in desired mutual amounts for a time suitable to lead to a homogenous mixture in any device known to be suitable to perform this operation.

In another aspect, the present invention relates to a method for the manufacture of a scented hard capsule as defined above, said method comprising a step (i) of contacting one or more capsules with a scenting composition as defined above.

As indicated above, it is preferred that the capsule is an empty hard shell capsule wherein shell caps and bodies have been pre-assembled in a non-permanently locked manner. This embodiment is preferred since it allows scenting the empty capsule without contaminating the capsule shell manufacturing equipment and also making the subsequent filling of the empty capsule easier due to the presence of the gliding enhancer agent in the scented composition.

Unless otherwise indicated, step (i) can be performed on a single capsule (i.e. each capsule is singularly contacted with the scenting composition) or on a batch of capsules (i.e. more than one capsules are simultaneously contacted). It is preferred that step (i) be performed on a batch of capsules.

In a preferred embodiment, before step (i) the method of the invention comprises a step (a) of preparing a scenting composition. In a preferred embodiment, step (a) is mixing together one of more scenting agents, one or more gliding enhancing agents and any other optional ingredient. Preferably, said step (a) can be performed according to any known technique in the art that is conventionally used for example in the pharmaceutical or cosmetic field to homogenously admix together powder and/or oil and/or liquid ingredients. Suitable mixing techniques are disclosed above.

In the embodiment wherein the scenting composition is in powder form and wherein capsules are contacted in batch, step (i) preferably comprises:
(i-1a) dusting a batch of capsules with the scenting composition of the invention, and subsequently to dusting
(i-2a) distributing the scenting composition over said batch of dusted capsules, e.g. tumbling, shaking, vibrating or agitating said batch, preferably by tumbling.

In the embodiment wherein the scenting composition is in oil form and wherein capsules are contacted in batch, step (i) preferably comprises:
(i-1b) depositing the scenting composition of the invention onto a batch of capsules, preferably by spray deposit or droplet deposit, and subsequently to depositing
(i-2b) distributing the scenting composition over said batch of dusted capsules, e.g. tumbling, shaking, vibrating or agitating said batch, preferably by tumbling.

Steps (i-2a) and (i-2b) can be performed for a time sufficient to obtain the desired homogeneity of scenting composition distribution in the batch of capsules.

When the capsules are empty hard capsules, the amount of scented composition to be used is advantageously chosen so as to have about between 25 and 100 ppm, preferably about between 50 and 75 ppm, more preferably about 50 ppm of composition per final scented capsule (ppm expressed in weight). Such amount of composition can be obtained by contacting about 1 g or less of the composition for 10 kg of empty capsules to be scented (wherein 10 kg are generally about 100 000 capsules depending on capsule dimension and weight), further taking into consideration that the amount of scented composition lost during the scenting method is generally minimal, typically less than 10% w/w of the total amount of composition contacted.

In the context of the method of the invention, the wording “capsule”, “scenting composition”, “scenting agents” and “gliding enhancing agents”, are as defined above for the capsules of the invention.

In another aspect, the present invention relates to the use of a scented hard capsule as defined above for the oral administration of one or more encapsulated products to a human or an animal being.

In another aspect, the present invention relates to the use of a scented hard capsule as defined above for the manufacture of a dosage form for oral administration of one or more encapsulated products to a human or animal being.

Suitable and preferred products to be encapsulated are one or more pharmaceutical and/or cosmetic agents. The substances to be encapsulated may be solids or liquids. The wording “pharmaceuticals pharmaceutical and/or cosmetic agents” encompasses for example chemical active principles (i.e. conventional drugs) as well as vitamins, probiotics, oligo-mineral complexes, plant extracts and mixtures thereof. The substances may be in solid form or in liquid form (e.g. water-based or lipid solutions, semi-solid formulations, microemulsions such as Smedds).

In the context of the uses of the invention, the wording “capsule”, “scenting composition”, “scenting agents” and “gliding enhancing agents”, are as defined above for the capsules of the invention.

Further advantages will become apparent to a skilled person by the disclosure of specific embodiments below.

### EXAMPLES

It is to be understood that the examples provided herein are illustrative only and not to be construed in a limiting sense.

### Example 1

### Hard HPMC Capsules Scented With Lemon Flavour Dissolved In Mineral Oil

Scented mineral oil was prepared by mixing 2 g of paraffin oil and 0.75 g of liquid lemon flavour. The scented oil so prepared was deposited on empty white opaque Vcaps® capsules—i.e. hard shell capsules obtained by a dip-moulding process using HPMC as film forming polymer and gellan gum as gelling agent. About 35 ppm of composition was deposited on each capsule. The scented capsule obtained showed good gliding properties, no visual defect, the glove test was negative. Capsules were stored in carton without specific closing. Smell test was positive at least 6 months after scenting and storing.

### Example 2

### Hard Shell Gelatine Capsules Scented With Strawberry Flavour Adsorbed On SLS

A strawberry flavoured SLS (containing 2 g of strawberry flavour per 10 g of powder SLS) was purchased (available from Firmenich-Geneva). The scented powder was dusted on a batch of empty hard shell gelatine capsules and the batch was subsequently tumbled to obtain repartition of the scented SLS.

About 55 ppm of composition was applied on each capsule. The scented capsule obtained showed good gliding properties, similar to standard non-scented hard gelatine capsules. No visual defect was observed. SLS powder was not visually detectable on capsule surface. Capsules were stored in carton without specific closing. Smell test was positive at least 6 months after scenting and storing.

### Example 3

### Hard HPMC Capsules Scented With Apricot Flavour Adsorbed On SLS

An apricot flavoured SLS (containing 50 mg of apricot per 150 mg of SLS) was purchased (available from Firmenich-Geneva). The scented SLS was dusted and tumbled on a batch of empty hard shell HPMC orange coloured capsules. About 55 ppm of composition was applied on each capsule. No interaction between the scented SLS and the capsule shell polymer was observed. The scented capsules were identical from a visible standpoint to standard non-scented capsules and exhibited the same properties of standard non-scented capsules.

### Example 4

### Red Opaque Hard Shell Gelatine Capsules Scented With Strawberry Flavour Dissolved In MCT

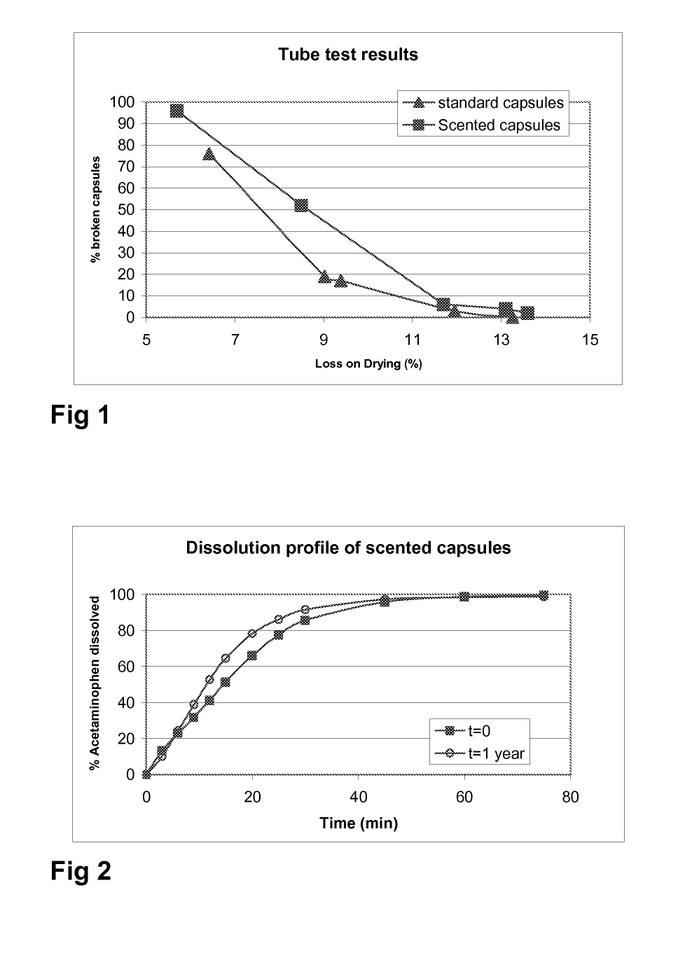

0.75 g of oil soluble strawberry was dissolved in 2 g of MCT. The scented oil was deposited on red opaque hard shell gelatine capsules. About 75 ppm of composition was applied on each capsule. Good gliding properties were observed, no visual defect, the glove test was negative. Capsules were stored in carton without specific closing. Smell test was positive at least one year after scenting and storing. The mechanical properties and the dissolution profile were tested after one year post scenting and they were found unchanged and comparable to those of standard non-scented capsules. In FIG. 1, mechanical properties of scented capsules and non-scented capsules are confronted. In FIG. 2, dissolution properties of scented capsules immediately after scenting and one year post scenting are confronted.

### Example 5

### Printed White Opaque Hard Shell HMPC Capsules Scented With Lime Flavour Dissolved In Paraffin

0.5 g of lime flavour was dissolved in 3 g paraffin oil. The perfumed oil was deposit on printed white opaque hard shell HMPC capsules. About 50 ppm of composition was applied on each capsule. No interaction between the scented composition, the capsule shell polymer and the ink was observed. The mechanical and physical properties of the scented capsules were not found different over the properties of non-scented capsules.

Based on the above, it can be appreciated that that advantages of the instant invention are for example:
the possibility to easily and effectively work with all type of film-forming polymers widely used in hard capsule manufacturing (e.g. gelatin, HPMC, pullulan, modified starches, derivatives thereof and mixtures thereof);
minimised risks of capsules shell modification since no solvent-like liquid is either used as dipping bath or sprayed onto capsules and no drying step is then necessary to remove the residual liquid. This removes the risk that capsule shells be either softened, deformed or made more brittle as in case of prior art technical solutions;
minimised risks of capsules shell chemical modification as chemical interactions between the scenting agents and the ingredients of capsules shells—namely, film-forming polymers—is reduced at minimum level. This removes for example the risk for polymer cross-linking that may take place in the shell upon adoption of conventional technical solutions and that deeply modify capsule dissolution/disintegration profiles;
minimised contamination of capsule production lines as the scenting agents may be added at a very final production step, in a physically separate environment, and e.g. just before packaging;
reduction of production costs as the invention allows reducing flavour in-line losses in light e.g. of a more accurate dosing of the scenting agents and the optimized homogeneity of flavour distribution on capsules surface

1. A scented hard capsule which is externally coated with a scenting composition, wherein said composition comprises one of more scenting agents and one or more gliding enhancing agents and wherein said composition is in powder form or oil form at about room temperature.

2. The capsule according to claim 1, wherein the scenting composition consists of one of more scenting agents and one or more gliding enhancing agents.

3. The capsule according to claim 1, wherein the capsule is an empty hard capsule.

4. The capsule according to claim 1, wherein said one or more scenting agents are present in an amount between 1 and 0.0001 weight parts per 1 weight part of the one or more gliding enhancer agents.

5. The capsule according to claim 4, wherein said one or more scenting agents are present in an amount between 0.02 and 0.5 weight parts per 1 weight part of the one or more gliding enhancer agents.

6. The capsule according to claim 1, wherein the capsule is an empty hard HPMC capsule.

7. The capsule according to claim 1, wherein the capsule is an empty hard gelatin capsule

8. The capsule according to claim 1, wherein the one or more gliding enhancing agents comprise SLS.

9. The capsule according to claim 1, wherein the one or more gliding enhancing agents comprise MCT, paraffin oil or mixtures thereof.

10. A method for the manufacture of a scented hard capsule comprising a step of contacting one or more capsules with a scenting composition, said composition comprising one of more scenting agents and one or more gliding enhancing agents and said composition being in powder or oil form at about room temperature.

11. The method of claim 10 wherein the capsules are empty hard capsules and the scented composition is applied so as to obtain between 25 ppm and 100 ppm of composition per final scented hard capsule.

12. A method of using a scented hard capsule for the manufacture of a dosage form for oral administration of one or more encapsulated products to a human or animal being.
